(19) United States
(12) Patent Application Publication
(10) Pub. No.: US 2002/0150633 A1
(43) Pub. Date: Oct. 17, 2002

(54) Dry dairy cow supplement

(76) Inventors: Myron Danzer, Glidden, IA (US); James Henry, Turlock, CA (US); Phillip Jardon, Carroll, IA (US)

Correspondence Address: FAY, SHARPE, BEALL, FAGAN, MINNICH & McKEE, Suite 700, 1100 Superior Ave., Cleveland, OH, 44114-2518, US

(21) Appl. No.: 10/095,291

(22) Filed: Mar. 11, 2002

Related U.S. Application Data
(63) Continuation-in-part of application No. 09/282,876, filed on Mar. 31, 1999, now Pat. No. 6,355,278.

(51) Int. Cl. A61K 35/70; A61K 35/78; A61K 33/18

(52) U.S. Cl. 424/687; 424/757; 424/195.16; 424/773

(57) ABSTRACT

A formulation for reducing a risk of developing hypocalcemia in dairy cattle comprises beet pulp, brewers grains or distillers grains, high temperature treated soybean meal having a protein dispersability index (PDI) of less than 10; hydrochloric acid; feed grade calcium carbonate and buffering compounds.

This application is a continuation-in-part of U.S. application Ser. No. 09/282,876 filed Mar. 31, 1999, now U.S. Pat. No. 6,355,278 dated Mar. 12, 2002.

### FIELD OF INVENTION

### BACKGROUND OF THE INVENTION

This invention relates to a range of formulations, the art of preparing same formulations and administering same formulations for inhibiting or reducing the risk of developing a disease which, in many cases, has a significant impact on the economics of dairy farming.

The disease is parturient hypocalcemia, commonly known as milk fever. This condition, to quote Ensminger, (“Dairy Cattle Science”, M. E. Ensminger, Third Edition (1993), Interstate Publishers, p. 287): “At or soon after calving (generally within 48 to 72 hours), a sharp decrease in blood calcium (hypocalcemia) occurs in some cows, resulting in a loss of appetite, subnormal temperature and an unsteady gait. This is followed by nervousness and, finally, collapse or complete loss of consciousness.” The triggering mechanism for the hypocalcemia (i.e., drop in blood calcium) is the onset of lactation which causes an intensive mobilization of calcium. Unless treated, milk fever can cause death and, therefore, the loss of the animal as a future milk producer.

The losses to the dairy farm economic sector can be significant. The total number of milk cows in the United States is in the order of 10 million (See, for example, Ensminger, op. Cit., p. 9), of which perhaps 60% are multiparous and at risk for milk fever. From a total milk production point of view alone, the 10 million milk cows represent about 1,500 billion pounds of milk in a given year. It is estimated that approximately 8% of all lactating cows are affected by clinical milk fever. It has also been estimated (Horst et. al, 1997) that the disease can reduce the productive life of a dairy cow by 3.5 years. Based upon its studies (C. Guard, Cornell Veterinary College, as reported in Hoard's Dairyman, January 1996), Cornell University further estimated that the average cost per milk fever case was $334. This value represented the direct cost of treating the clinical cases and the estimated production losses of milk. Multiplying the number of potential cases by the $334 per case yields an estimated total loss to the U.S. dairy industry of $160 million per year. Another factor affecting economic losses is that milk cows contracting milk fever are also more susceptible to a host of secondary problems, which include ketosis, mastitis, dystocia, retained placenta, displaced abomasum and uterine prolapse (Horst et al., 1997). Each of these secondary problems have, as the least consequence, a reduction or further reduction of milk production.

At the subclinical level, a much higher percentage of dairy cows are affected; as many as 50 to 60% of older cows in some herds are affected. The diseases associated with subclinical hypocalcemia include retained placenta, displaced abomasums, ketosis and mastitis.

Since milk fever has such a major impact on the economy of dairy farming in the United States and elsewhere in the world, a considerable amount of research has been directed towards inhibiting its effects and reducing the risk of contracting the disease.

Perhaps the most significant early research was reported by a group of Norwegian researchers (Ender, F. and I. W. Dishington, 1967, “Comparative Studies on Calcium Balance Levels in Parturient Cows Fed Diets Inducing and Preventing Milk Fever.” 557 Am. XVIIIth World Veterinary Congress, Paris, France). These researchers demonstrated that the effect of various cations in dietary rations of a dairy cow, in terms of promoting milk fever incidence, could be prevented by adding anions in the form of hydrochloric or sulfuric acid. However, they realized that with the other feed ingredients at their disposal at the time, using these highly corrosive acids in their un-reacted forms would pose a serious hazard to both dairy cattle and dairy farm personnel. Thus, these researchers concluded that, instead of these pure acids, related anionic salts such as calcium chloride, ammonium sulfate, aluminum sulfate and magnesium sulfate should be considered.

Based upon these findings, other researchers (most prominently, Goff and Horst then started evaluating these anionic salts. They found that, while these salts are relatively easy to handle, there are problems with palatability and the intrinsic problem that, while salts contain anions, they also contain cations, which depending upon their rate of absorption, will negate some of the positive effects of the anions.

The same paper (J. R. Goff and R. L. Horst, “Using Hydrochloric Acid as a Source of Anions for Prevention of Milk Fever,” USDA Prepublication (1997)) compares hydrochloric acid with the anionic salt, calcium chloride and shows the advantage of the former over the latter. The authors also discuss in some detail comparative feeding studies involving dairy cow diets with or without hydrochloric acid to control milk fever. In a subsequent paper, (“Use of Hydrochloric Acid as a Source of Anions for Prevention of Milk Fever,” J. Dairy Science, November 1998, p. 2874-2880), Goff and Horst provide further evidence for their earlier conclusions.

While this paper is substantive in its proof that, to quote the authors, “hydrochloric acid is an inexpensive, ‘palatable’ alternative to anionic salts as a means of controlling milk fever,” the palatability is only improved as compared to those of anionic salts. Hydrochloric acid used with the formulations studied is still difficult to administer, as the dairy cow does not want to consume it in the quantities required.

Because of the problems mentioned with respect to inorganic acids, past research was focused on preparing formulations using anionic salts and not those using hydrochloric or sulfuric acids.

For example, Rebhan (Herbert J. Rebhan, U.S. Pat. No. 4,931,290, “Milk Fever Prophylactic Treatment and Composition”) describes a method for “reducing the propensity of a dairy cow to develop severe milk fever upon calving comprising of administering thereto a composition (consisting) of a water-soluble calcium compound and complexing agent for serum phosphorus.” That patent discusses the use of (anionic) calcium salts of hydrochloric and organic acids supplemented with a complexing agent to form water-insoluble compounds of serum phosphorus. The combinations of these compounds are presented as having a favorable effect on the blood anion-cation balance thereby inhibiting the effects or reducing the chance of contracting milk fever.

A further example by Kjems (Gunnar Kjems, U.S. Pat. No. 5,393,535, “Orally Administratable Calcium Supplement for Cattle”) describes a composition wherein a calcium ion (i.e., an anionic salt) is dissolved in water but the water phase is dispersed into a continuous oil phase forming an emulsion by means of a non-ionic emulsifier. This method of preparation is presented as compatible with oral administration of the composition and as palatable to the dairy cow because of the oily phase.

In another example, Goff and Horst (Jesse P. Goff and Ronald L. Horst, U.S. Pat. No. 5,560,920, “Calcium Formulations for Prevention of Parturient Hypocalcemia”) present that calcium propionate (i.e. an anionic organic salt) may be mixed with propylene glycol and either citric or phosphoric acid to form a non-hardening paste or with sodium chloride to form a liquid drench. It is stated that these formulations are particularly effective in treating the hypocalcemia associated with the onset of lactation in dairy cows. The inventors argue that calcium-containing gels can be made with water-soluble carriers (as discussed by Kjems) such as oils or with water-soluble carriers such as propylene glycol.

They state that the oils tend to decrease the availability of calcium for absorption, but that the gels formed with propylene glycol are more readily soluble in water and thereby increase the availability of calcium.

In still another example, Abele (Ulf Abele, U.S. Pat. No. 5,631,289. “Use of Calcium Formate in Orally Administrable Compositions”) discusses the advantages of an anionic salt of formic acid (i.e. calcium formate) in the prophylaxis and metaphylaxis of calcium deficiency in dairy cows. It is stated that the absorption of calcium is similar for either calcium formate or calcium chloride, but that the former is not corrosive or irritating to the membranes of the digestive tract and thus it is both more palatable and is also less dangerous to the animal if per chance the formulation is inhaled in the respiratory tract, as compared to calcium chloride formulations. Even so, the patent recommends that the calcium formate be bound in a gel or paste to further improve palatability and further lessen the chance of ingestion into the respiratory tract.

The background material presented above summarizes current knowledge. That knowledge may also be formulated as follows:

To reduce the risk of developing hypocalcemia, it is important that the dry cow has the proper anion-cation balance in its body fluids. There are a number of equations referred to as “dietary cation-anion difference equations,” which describe the effect diet cations and anions will have on the blood and urine pH of the dry cow. These equations show the dietary effects of the individual ions, such as the cations sodium, potassium, calcium and magnesium and the anions chlorine, sulfur and phosphorus. One of the dietary cation-anion equations (DCAD) is based upon the research performed by Goff (Cation-Anion Difference of Diets & Its Influence on Milk Fever, J. P. Goff, USDA. Prepublication (1998), 14 pages) and is expressed as: (Na(+) +K(+)+0.15Ca(++)+0.15Mg(++))−Cl(−)+0.25S(−−)+0.5P(−−−)). This is a state-of-the-art equation describing the effect diet cations and anions will have on the blood and urine pH of dry cows.

Hypocalcemia in dry cows manifests itself, other than by showing the previously described symptoms, by concentrations of anions and cations in the blood and urine which overall have an alkalinizing effect. Depending upon the forages fed, more or less dietary sodium and potassium may enter the blood stream and ultimately the urine stream. While dietary sodium and potassium should be kept as low as possible, other anions such as chloride may need to be added to cause a large enough negative dietary cation-anion difference in the body fluids to, in turn, cause a desirable acidic pH in the urine.

As stated earlier, sodium, and particularly potassium, levels should be kept as low as possible. However, depending upon the forages available, this cannot always be achieved. There are also limits on how much phosphorus, magnesium and sulfate should be contained in the diet. Another variable is the calcium intake. While it would be likely that more calcium in the cow's intestinal system would better allow the cow to utilize intestinal calcium absorption as well as bone calcium reabsorption to prevent hypocalcemia, there are limits as to how much calcium should be fed. Too high a percentage in the feed could affect palatability and thus reduce feed intake and could also increase the alkalinizing activity. Thus, the major variable that can be manipulated to reach a desirable negative DCAD is the chloride content of the diet. A desirable range for the DCAD is typically −800 to −2,200 meq/kg.

There are, however, limits to the addition of chloride. In most cases, the cows will tolerate a 0.8 to 1.0% by weight of chloride based upon the total dairy ration fed. However, in most cases, cows will find diets containing more than 0.8% by weight of chloride less palatable, thus, limiting the intake of the ration.

### SUMMARY OF THE INVENTION

The invention presented herein relates to formulations for reducing the risk of developing hypocalcemia in dry dairy cows, methods for preparing same and the use of guidelines for the effective use of the formulations.

The inventors in their earlier U.S. Pat. No. 6,355,278 discussed their formulations for reducing the risk of developing hypocalcemia. These formulations use hydrochloric acid as a source of anions and a high bypass protein soybean meal as a carrier for the acid. The high bypass protein soybean meal is prepared according to the methods presented in their U.S. Pat. No. 5,225,230 and is characterized by a protein dispersability index (PDI) of less than 10 and a bypass value of 58 to 65% by weight of the protein.

To further develop the formulations, the inventors focused on improvements in the use of hydrochloric acid as a supplement to the formulations. One criterion was the need to impart on the formulations a negative DCAD within the range of −800 to −2,200 meq/kg. These formulations contain more or less anions depending upon the make-up of the formulation and the balance of the diet. To determine whether more or less anions are needed, monitoring the urine pH is the best method. A typical target is a urine pH of 6.2 to 6.7 in Holstein cows. To further improve on the formulations, studies were done to determine the optimum pH level of these formulations.

The initial formulations prepared according to the methods of U.S. Pat. No. 6,355,278 had low pH levels, generally below 2. Researching the optimum pH levels of the formulations showed that a pH range of 2.5 to 4.5 was optimum in two respects. An increase of the pH above the range resulted in a less palatable product. Decreasing the pH below 2.5 caused several kinds of problems. It made it more difficult for the dairy farmer to handle while preparing a mixture of the formulation and the balance of the feed ration, because of the acidity of the formulations and the hydrochloric acid odors. In addition, the highly acidic formulations could cause irritation of the intestinal membranes of the cow and if dust and vapors from the formulations was inhaled, it could cause problems in the cow's respiratory tract.

In order to control the acidity of the formulations at the desired pH level, buffers were introduced to control same. Calcium carbonate and magnesium oxide were added as buffers and it was found that pH levels in the formulations could be lifted from a pH of 2 or less to the desired level of 2.5 to 4.5. An additional advantage of buffering the pH of the formulations was the lowering of the vapor pressure of the hydrochloric acid component, resulting in improvements in the preparation process of the formulations and in the handling of these formulations. For example, in the manufacturing process the formulations could be dried without flashing off hydrochloric acid vapors. The lesser vapor pressure also improved handling by dairy farm workers, as the dust of these formulations was less irritating to the eyes and the respiratory tract.

Other chemical compounds that may be used as buffers, for example, include oxides, hydroxides, carbonates and bicarbonates.

The inventors found that the buffering process could be accelerated by using wet instead of dry distillers grains or dry brewers grains. The presence of moisture aids in the migration of the buffering compounds. A further advantage is that the moisture also helps to dissipate the heat of the reactions.

The inventors also found that further improvements to the formulations could be made by reducing sodium and potassium in the feed ration. Instead of using the common grade of distillers grains which is high in sodium and potassium, a distillers grain with those solubles extracted in beneficial to use. Also, instead of using the common grade of beet pulp which contains molasses high in sodium and potassium, a beet pulp with a substantially lesser content of molasses is beneficial to use.

The research performed by Goff (USDA, Ames, Iowa) provides the following guidelines for the use of the formulations of this invention for reducing the risk of developing hypocalcemia:

a) Reduce the diet potassium and sodium levels to the required levels of these nutrients suggested by the National Academy of Sciences publication “Nutrient Requirements of Dairy Cattle 2001.”

b) Monitor the urine pH during the pre-parturient period. If the urine pH is found to exceed 7, add a quantity of a formulation as described earlier to reduce urine pH to the level of 6.0 to 7.0. The desired quantity is typically 2 to 4 lbs. per cow per day, depending upon the weight of the cow, the potassium and sodium content of the ration utilized and the specific formulation used.

c) Adequacy of the amount of a specific formulation added to the ration can be assessed three days after addition of the formulation to the ration by determining if an adequate decrease in urine pH has occurred. The quantity of the specific formulation can be increased if the urine pH is still in excess of 7.0. The quantity of the specific formulation added to the ration can be decreased if urine pH is reduced below 6.0. Once the proper amount of the specific formulation has been added to the ration to achieve the desired urine pH level of 6.0 to 6.7, the cow should be maintained on the diet containing the specific formulation for a period of at least five days and up to 28 days prior to calving.

### BRIEF DESCRIPTION OF THE DRAWINGS

The invention may take physical form in certain parts and arrangement of parts, a preferred embodiment of which will be described in detail in this specification and illustrated in the accompanying drawings.

FIGS. 1 and 2 are block diagrams illustrating the steps in the practice of the present invention for producing formulations consisting of a soybean meal prepared as described in U.S. Pat. No. 5,225,230, hydrochloric acid, buffers and other ingredients.

### DETAILED DESCRIPTION OF THE INVENTION

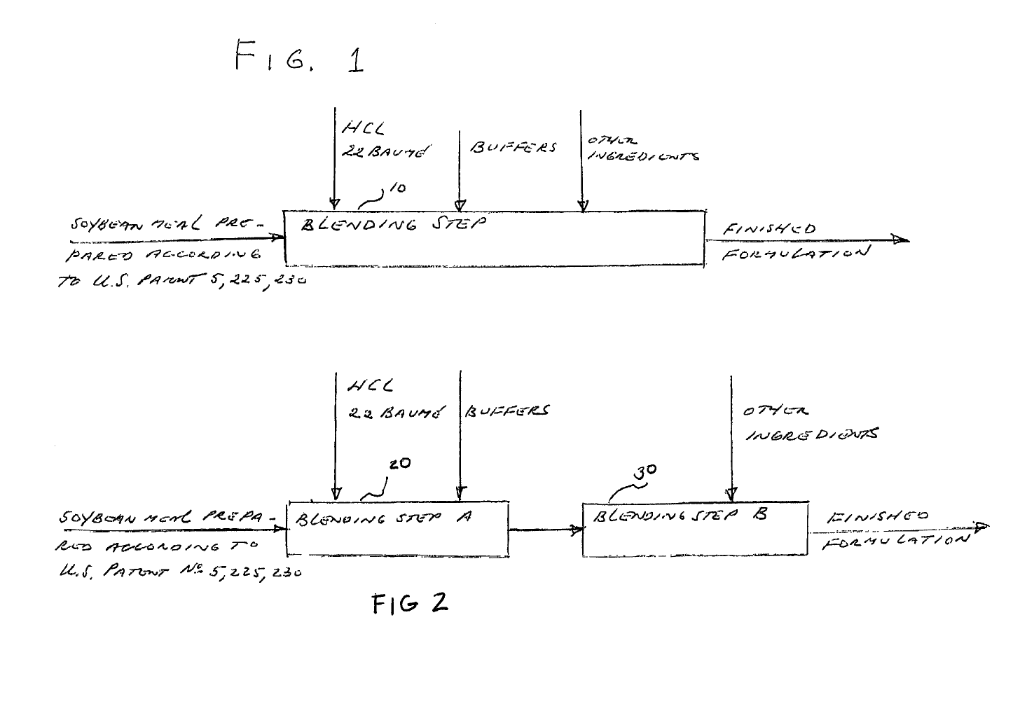

Referring now to the drawings, wherein the showings are for the purpose of illustrating the preferred embodiments of the invention only and not for the purpose of limiting same, FIGS. 1 and 2 are block diagrams showing the step or steps followed in practicing the present invention. In particular, the figures set forth the step or steps of blending soybean meal prepared according to the methods described in U.S. Pat. No. 5,225,230 with a hydrochloric acid of a desired Baume strength, buffers and other ingredients.

Block 10 on FIG. 1 shows the blending step in which the soybean meal is introduced into a stirred vessel together with hydrochloric acid, buffers and other ingredients.

Blocks 20 and 30 on FIG. 2 show an alternative blending step in which the ingredients are admixed sequentially (i.e. one or more ingredients are added after two or more of the other ingredients have been blended).

Blending is performed in batch mode. Weighted quantities of soybean meal, hydrochloric acid, buffers and other components are entered in the blending vessel wherein an agitating arm stirs the solids and liquid for a time period sufficient to insure homogeneity of the formulation, complete absorption of the hydrochloric acid into the soybean and other components and buffering of the hydrochloric acid. The completeness of the blending is typically achieved by agitating for a period of 15 to 30 minutes.

Soybean meal prepared according to the methods of U.S. Pat. No. 5,225,230 was introduced into a 10 ft long, 4 ft wide and 5 ft deep ribbon blender in which approximately 1,000 lbs. of soybean meal was blended with 1,000 lbs. of hydrochloric acid with a strength of 22 Baume, 2,000 lbs. of beet pulp, 3,000 lbs. of dried brewers' grains or distillers dried grains, 330 lbs. of feed grade calcium carbonate and 160 lbs. of feed grade magnesium oxide. The resulting mixture is available under the commercial trademark, SoyChlor 37 (SoyChlor-registered Trademark), and its blended crude protein content is 37%. This formulation is characterized by a negative dietary cation-anion difference from minus 800 to minus 1,600 meq/kg. Required feed rate may be typically 2 to 4 lbs. per day.

In still another example, 410 lbs. of the soybean meal was blended with 820 lbs. of hydrochloric acid with a strength of 22 Baume, 980 lbs. of beet pulp, 1,640 lbs. of dried brewers grains or distillers dried grains, 160 lbs. of feed grade calcium carbonate and 70 lbs. of feed grade magnesium oxide were blended to produce approximately 3,260 lbs. of formulation following 30 minutes of blending. This product is commercially available as SoyChlor 16 (SoyChlor-registered Trademark) as its blended crude protein is 16% by weight. This formulation is characterized by a negative dietary cation-anion difference range from minus 1,200 to minus 2,200 meq/kg. Required feed rate may be 2 to 4 lbs. per day.

In yet another example, 380 lbs. of the soybean meal was blended with 570 lbs. of hydrochloric acid with a strength of 22 Baume, 1530 lbs. of dried brewers grains or distillers dried grains, 890 lbs. of beet pulp, 130 lbs. of feed grade calcium carbonate, 150 lbs. of feed grade magnesium oxide, 300 lbs. of calcium chloride and 50 lbs. of magnesium silicate to produce approximately 4,000 lbs. of formulation which is commercially available as SoyChlor 16-7. As above, this formulation has a crude protein content of 16% by weight. This formulation has a negative dietary cation-anion difference in excess of minus 2,000 meq/kg and its increased chloride content, from both sources (i.e., the hydrochloric acid and the calcium chloride) allow it to be effective at feed rates of 1 to 2 lbs. per day.

What is claimed is:

1) A formulation for reducing the risk of developing hypocalcemia in dairy cattle comprised of:
a) Two to three parts by weight of beet pulp;
b) Three to five parts by weight of dried brewers grains or distillers grains;
c) One part by weight of a high temperature treated soybean meal characterized by having a protein dispersability index (PDI) of less than 10;
d) One-and-one-half to two parts by weight of hydrochloric acid solution in the strength of 22 Baume;
e) One third of a part by weight of feed grade calcium carbonate; and
f) Buffering compounds such as oxides, hydroxides, carbonates and bicarbonates, compatible with the nutrition and health needs of dairy cattle for the purpose of:
a) maintaining the pH of the formulation in the range of 2.5 to 4.5; and
b) maintaining a negative dietary cation-anion difference (DCAD) of at least minus 800 to minus 1,600 meq/kg of the formulation subject to the following limitations:
a) phosphorus 40 to 50 grams per cow per day for the total diet, i.e. both the formulation and the balance of the diet;
b) magnesium 0.3% to 0.5% by weight of the total daily diet, i.e. both the formulation and the balance of the diet; and
c) sulfate not to exceed 0.4% by weight of the total daily diet, i.e. both the formulation and the balance of the diet.

2) A formulation according to claim 1 wherein magnesium is added in the form of magnesium oxide at the quantity of one sixth to one third parts by weight.

3) A formulation for reducing the risk of developing hypocalcemia in dairy cattle comprising a blend of:
a) Two to two-and-one half parts by weight of beet pulp;
b) Four parts by weight of dried brewers grains or distillers grains;
c) One part by weight of a high temperature-treated soybean meal characterized by a PDI of less than 10;
d) Two parts by weight of hydrochloric acid in the strength of 22 Baume;
e) One-third to one-half of a part by weight of feed grade calcium carbonate; and
f) Buffering compounds such as oxides, hydroxides, carbonates, and bi-carbonates compatible with the nutrition and health needs of dairy cattle for the purpose of:
a) Maintaining the pH of the formulation in the range of 2.5 to 4.5; and
b) Maintaining a negative DCAD of minus 1200 to minus 2200 meq/kg of the formulation subject to the following limitations:
a) Phosphorus, 40 to 50 grams per cow per day in the total daily diet, i.e. both the formulation and the balance of the diet;
b) Magnesium, 0.3% to 0.5% by weight of the total daily diet, i.e. the formation and the balance of the diet; and
c) Sulfate, not to exceed 0.4% by weight of the total daily diet, i.e. the formulation and the balance of the diet.

4) The formulation of claim 3 wherein one-sixth to one-third parts by weight of magnesium oxide is added to the formulation.

5) The formulation of claim 3 wherein quantities of wet distillers or wet brewers grains are used instead of dried products, such that the compositions on a dry basis do not change.

6) The formulations of claim 3 wherein solubles containing sodium and potassium have been extracted.

7) The formulation of claim 3 wherein the beet pulp is without the molasses fraction so as to reduce a sodium and potassium content of the ration.

8) A formulation for reducing the risk of developing hypocalcemia in dairy cattle comprising a blend of:
a) Dried brewers grains or distillers dried grains: 38.25% by weight of the formulation;
b) Beet pulp: 22.25% by weight of the formulation;
c) High temperature-treated soybean meal characterized by a PDI of less than 10: 9.5% by weight of the formulation;
d) Hydrochloric acid 36% solution: 14.25% by weight of the formulation;
e) Calcium chloride 48%: 7.5% by weight of the formulation;
f) Magnesium oxide 54%: 3.75% by weight of the formulation;
g) Feed grade calcium carbonate: 3.25% by weight of the formulation; and
h) Magnesium silicate: 1.25% by weight of the formulation.

9) The formulation of claim 8 wherein quantities of wet distillers or wet brewers grains are used instead of dried products, such that the compositions on a dry basis do not change.

10) The formulation of claim 8 wherein solubles containing sodium and potassium have been extracted.

11) The formulation of claim 8 wherein the beet pulp is without the molasses fraction so as to reduce a sodium and potassium content of the ration.

12) Method for producing a formulation for reducing the risk of developing hypocalcemia in dairy cattle comprising of the following steps:
a) Preparing a high temperature-treated soybean meal characterized by a PDI less than 10;
b) Adding a hydrochloric acid solution;
c) Adding feed ingredients other than soybean meal; and
d) Adding buffering compounds such as oxides, hydroxides, carbonates, and bicarbonates compatible with the nutrition and health needs of dairy cattle for the purpose of:
a) maintaining the pH of the formulation in the range of 2.5 to 4.5; and
b) maintaining a negative dietary cation-anion difference (DCAD) of at least minus 800 to minus 2,200 meq/kg of the formulation subject to the following limitations:
a) Phosphorus, 40 to 50 grams per cow in the total daily diet, i.e. formulation and the balance of the diet;
b) Magnesium, 0.3% to 0.5% by weight of the total daily diet, i.e. formulation and balance of the diet; and
c) Sulfate, not to exceed 0.4% by weight of the total daily diet, i.e. the formulation and the balance of the diet; and
e) Blending the above ingredients.

13) A method according to claim 12 in which the high temperature treated soybean is prepared as follows:
a) comminuting the soybeans to crack open their hulls and shatter their kernels;
b) heating the comminuted soybeans in a high temperature reactor to elevated temperatures in the range of 235 to 350 degrees Fahrenheit;
c) maintaining the soybeans at elevated temperatures for a period of 1 to 60 minutes;
d) partially removing the oil contained in the soybeans by mechanical means; and
e) grinding the soybean cake produced by mechanical means to produce a soybean meal with a particle size range of 1000 to 1200 microns.

14) A method according to claim 13 including the additional step of:
adding one to two parts by weight of hydrochloric acid solution with a strength of 22 Baume.

15) A method according to claim 14 including the additional step of:
adding 2 to 3 parts by weight of beet pulp and 3 to 5 parts by weight of dried brewers grains.

16) A method according to claim 15 including the additional step of:
adding one-third to one-half part by weight of feed grade calcium carbonate.

17) A method according to claim 16 including the additional step of:
adding one-sixth to one-third part by weight of magnesium oxide.

18) A method according to claim 17 including the additional step of:
adding one-tenth to two-tenths part by weight of magnesium silicate.

19) A method for reducing the risk of developing hypocalcemia in dairy cows, comprising the steps of:
a) Reducing diet sodium and potassium as close as possible to the required levels suggested by the National Academy of Science publication, “Nutrient Requirements for Dairy Cattle 2001;”
b) Monitoring urine pH in the pre-parturient period to establish whether it is at 7.0 or above;
c) If the urine pH is found to exceed 7.0, adding a quantity of a formulation as defined in claim 1 to the feed ration for the purpose of reducing urine pH to the level of 6.0 to 7.0;
d) Three days subsequent to the introduction of a formulation as defined in claim 1 to the ration, determining whether an adequate decrease of urine pH has occurred;
e) Increasing a quantity of the formulation added if urine pH is still in excess of 7.0;
f) Decreasing a quantity of the formulation if urine pH is reduced to below 6.0; and
g) Once a proper amount of the formulation has been added to the ration to achieve the desired urine pH level of 6.0 to 6.7, maintaining the cow on the diet including the formulation for a period of at least 5 days and up to 28 days prior to calving.
